(19) United States
(12) Patent Application Publication
(10) Pub. No.: US 2022/0099202 A1
(43) Pub. Date: Mar. 31, 2022

(54) Fluid Unidirectional Flow Structure, Check Assembly, and Respiratory Device

(71) Applicant: KUNSHAN YUANSHAN TIANDI SOFTWARE TECHNOLOGY CO., LTD., Jiangsu (CN)

(72) Inventor: Yixin Sun, Shanghai (CN)

(21) Appl. No.: 17/434,371

(22) PCT Filed: Apr. 10, 2020
(86) PCT No.: PCT/CN2020/084146

§ 371 Date: Aug. 26, 2021

(30) Foreign Application Priority Data
Feb. 26, 2019 (CN) 201910144542.9

(51) Int. Cl. F16K 15/14; F16K 3/06; A62B 9/02; F16K 3/314; F16K 15/02

(52) U.S. Cl. CPC F16K 15/144; F16K 3/06; F16K 15/02; F16K 3/314; A62B 9/02

(57) ABSTRACT

Disclosed is a fluid unidirectional flow structure. The fluid unidirectional flow structure (100) comprises; a first flow-checking body (110) and a second flow-checking body (120); the first flow-checking body (110) comprises a first connection portion (111) that is interconnected with a first flow portion (112) having at least one first through-hole (113); the second flow-checking body (120) comprises a second connection portion (121) interconnected with a second flow portion (122) having at least one second through-hole (123); when a fluid reverses directions, at least part of the first flow portion (112) moves relative to at least part of the second flow portion (122). The fluid unidirectional flow structure (100) may be designed in any shape, so as to facilitate installation. Also disclosed are a check assembly, and a respiratory device, when used on a breathing passageway, the invention may be adapted to a respiratory passageway of any size and shape. The fluid unidirectional flow structure (100) may also be installed on the body of a respiratory isolation mask (1000), wherein the shape of said mask may be adapted to the shape of the face of a person or animal. The shape of the first flow-checking body (110) and of the second flow-checking body (120) may be configured to be adaptable, so as to better bring into play the function and utility of the fluid unidirectional flow structure.

### CROSS-REFERENCE TO RELATED APPLICATION

The present disclosure claims the priority to the Chinese patent application with the filing number CN201910144542.9, filed on Feb. 26, 2019 with the Chinese Patent Office, and entitled “Fluid Unidirectional Flow Structure, Check Assembly, and Respiratory Device”, the contents of which are incorporated herein by reference in entirety.

### TECHNICAL FIELD

The present disclosure relates to the field of mechanical devices, and in particular, to a fluid unidirectional flow structure, a check assembly, and a respiratory device.

### BACKGROUND ART

The unidirectional flow structure is a structure in which a fluid can only flow from an inlet but cannot flow back, for example, a unidirectional valve, but the unidirectional valve in the prior art has a larger volume and occupies a large space, and particularly when a cross section is of an irregular shape, it is not easy to install.

### SUMMARY

An objective of embodiments of the present disclosure lies in providing a fluid unidirectional flow structure, a check assembly, and a respiratory device, aiming at overcoming the problems that the exiting unidirectional valves occupy a large space and are not easy to install.

An embodiment of the present disclosure provides a fluid unidirectional flow structure, which fluid unidirectional flow structure includes a first flow-checking body and a second flow-checking body;

the first flow-checking body includes a first connection portion and a first flow portion connected to each other, and the first flow portion is provided with at least one first through-hole;

the second flow-checking body includes a second connection portion and a second flow portion connected to each other, and the second flow portion is provided with at least one second through-hole;

when the fluid flows in a different direction, at least a part of the first flow portion and at least a part of the second flow portion are capable of moving relative to each other;

when the fluid flows forward, a gap is formed between the first flow portion and the second flow portion, and the gap is in communication with the at least one first through-hole and the at least one second through-hole; and

when the fluid flows reversely, all of the first through-holes are blocked by the second flow-checking body, and/or all of the second through-holes are blocked by the first flow-checking body.

Optionally, the first through-holes and the second through-holes are arranged in a manner of being staggered with each other.

The fluid unidirectional flow structure may be designed in an arbitrary shape according to the material and shape of the flow guide pipe, so as to facilitate installation.

In an embodiment of the present disclosure, when the fluid unidirectional flow structure is applied to a respiratory device, for example, installed to an inhalation tube or a respiratory/breathing tube, the fluid unidirectional flow structure is used for opening and closing during the inhalation and exhalation, which may avoid exhaled gas from being inhaled again during the inhalation, and a supply gas goes into mouth and nose all the time during the inhalation.

Respiratory tubes have different shapes and different dimensions, but the fluid unidirectional flow structure may be applicable to all cases. The fluid unidirectional flow structure may also be installed on a respiratory/breathing isolation mask body, the respiratory isolation mask has a shape adapted to a face shape of a person or an animal, and the first flow-checking body and the second flow-checking body may be set to adapted shapes, so as to better exert the function and effect of the fluid unidirectional flow structure.

Optionally, when the fluid flows reversely, two opposite surfaces of the first flow portion and the second flow portion are partially attached to each other, there is a gap between remaining portions of the two surfaces, and the gap is in communication with none of the first through-holes and none of the second through-holes.

Due to factors such as stress of the fluid and the shape of the first flow portion, the first flow-checking body and the second flow-checking body may be formed into arbitrary shapes in the relatively narrow and compact flow guide pipe, and when the fluid flows reversely, the two opposite surfaces of the first flow portion and the second flow portion may not be completely coincidentally attached to each other, but it is feasible as long as the first through-holes and the second through-holes are not in communication with each other.

In an embodiment of the present disclosure,

optionally, the first connection portion is provided at an outer edge of the first flow portion, and the second connection portion is provided at an outer edge of the second flow portion.

The fluid cannot flow through the first connection portion or the second connection portion; and under the effect of the fluid, the first flow-checking body and the second flow-checking body are stressed more uniformly. Besides, the first connection portion and the second connection portion are provided around the first flow portion and the second flow portion, respectively, facilitating the installation of the fluid unidirectional flow structure in the flow guide pipe, without a gap therebetween.

In an embodiment of the present disclosure,

optionally, the first flow-checking body and the second flow-checking body are capable of sliding relative to each other, and when the fluid flows in a different direction, the first flow portion and the second flow portion are capable of being attached to or separated from each other.

In an embodiment of the present disclosure,

optionally, when the fluid flows reversely, two opposite surfaces of the first flow portion and the second flow portion are at least partially attached to each other; and the two surfaces are both curved surfaces or planes.

The two surfaces are both curved surfaces or both planes, there is no gap between attached parts of the two surfaces, then the sealing performance may be stronger when stopping flowing.

In an embodiment of the present disclosure,

optionally, an elastic modulus of the first flow portion is greater than that of the second flow portion; and

optionally, the first flow portion is made of a rigid material, and the second flow portion is made of a flexible material. The second flow portion, being flexible, may be deformed, and under the effect of the fluid, the second flow portion is deformed to a greater degree to achieve the effect of opening and closing, and occupies a small space.

In an embodiment of the present disclosure,

optionally, the fluid unidirectional flow structure further includes an adjusting member;

the adjusting member is movably connected to the first flow-checking body, and a part of cross section of at least one first through-hole is covered by the adjusting member; alternatively

the adjusting member is movably connected to the second flow-checking body, and a part of cross section of at least one second through-hole is covered by the adjusting member.

The adjusting member can cover a part of the first through-holes or a part of the second through-holes, or may cover all the first through-holes or all the second through-holes. The adjusting member therefore can adjust the flow rate of the first flow portion or the second flow portion.

When the fluid unidirectional flow structure is applied to a respiratory device, the adjusting member may adjust a volume of the supply gas entering the nose and mouth, for example, all the first through-holes are opened during exercise, and an amount of gas is adjusted to a maximum, and the amount of gas may be reduced when sitting or lying down. It is convenient to make adjustment, and the comfort degree is increased.

In an embodiment of the present disclosure,

optionally, the adjusting member is capable of sliding towards the first flow-checking body to be attached to the first flow-checking body, and the adjusting member is capable of sliding away from the first flow-checking body to be separated from the first flow-checking body; alternatively

optionally, the adjusting member is capable of being rotated about an axis passing through a centroid of a cross section of the first flow-checking body transverse to a flowing direction of the fluid, so as to cover a part of the cross section of the first through-hole.

Optionally, the adjusting member is rotatably connected to a side of the first flow-checking body away from the second flow-checking body and closely attached to a surface of the first flow-checking body.

An embodiment of the present disclosure provides a check assembly, wherein the check assembly includes a flow guide pipe and the fluid unidirectional flow structure provided in an embodiment of the present disclosure; the fluid unidirectional flow structure is installed inside the flow guide pipe, the fluid unidirectional flow structure divides the flow guide pipe into a first chamber and a second chamber; and the fluid unidirectional flow structure is capable of making the first chamber isolated from or in communication with the second chamber.

The check assembly has all the advantages of the fluid unidirectional flow structure. In the check assembly, the fluid unidirectional flow structure is provided inside the flow guide pipe, facilitating the installation of the fluid unidirectional flow structure. With regard to the flow guide pipe with an irregular cross section, the fluid unidirectional flow structure also may be directly installed, without the need of procedures such as cutting the flow guide pipe such that the irregular cross section is divided into regular cross sections, which is more suitable for more scenarios.

Optionally, the first flow-checking body is fixedly connected in the flow guide pipe, and the second flow-checking body is slidably connected in the flow guide pipe; alternatively,

optionally, the first flow-checking body and the second flow-checking body are both fixedly connected in the flow guide pipe, and the first flow-checking body and the second flow-checking body have different elastic moduli.

Optionally, an outer edge of the first flow-checking body is closely attached to an inner wall of the flow guide pipe.

Installation is facilitated. When the fluid unidirectional flow structure is closed, the fluid can be avoided from flowing between the edge of the first flow-checking body and the flow guide pipe, and other components such as the sealing assembly are omitted.

Optionally, the flow guide pipe is provided therein with a slide rail or a slide groove having a preset length; and the second flow-checking body is slidably connected to the slide rail or the slide groove.

Optionally, the flow guide pipe is provided therein with a limiting block protruding in a radial direction, the first flow-checking body is fixedly connected to the flow guide pipe, the second flow-checking body is provided between the limiting block and the first flow-checking body, and the second flow-checking body is capable of sliding between the limiting block and the first flow-checking body.

When the fluid flows in a different direction, the first flow-checking body and the second flow-checking body are enabled to slide relative to each other, due to the change of direction of a force applied on the first flow-checking body and the second flow-checking body after the fluid flows in the different direction (i.e. flowing direction of the fluid is changed), so as to achieve the purpose that at least a part of the first flow portion and at least a part of the second flow portion can move relative to each other.

An embodiment of the present disclosure provides a check assembly, wherein the check assembly includes a flow guide pipe and the fluid unidirectional flow structure provided in an embodiment of the present disclosure; and an outer wall of the flow guide pipe is provided with an installation hole running through the flow guide pipe, and the first flow-checking body is installed in the installation hole.

The installation hole runs through the outer wall of the flow guide pipe, and the fluid unidirectional flow structure controls whether the inside and outside of the flow guide pipe are in communication with each other, and further control whether to discharge the fluid inside the flow guide pipe, or prevent the fluid outside the flow guide pipe from flowing into the flow guide pipe.

When the check assembly is used in a respiratory device, for example, the check assembly is installed in an inhalation tube, the flow guide pipe acts as the inhalation tube, and the fluid unidirectional flow structure is opened during exhalation, the gas exhaled is discharged by the flow guide pipe, and the fluid unidirectional flow structure is closed during inhalation, and the outside gas will flow into the mouth and nose only through the flow guide pipe.

Optionally, the first connection portion is hermetically connected to the inner wall of the flow guide pipe.

Installation is facilitated. When the fluid unidirectional flow structure is closed, the fluid may be avoided from flowing between the edge of the first flow-checking body and the flow guide pipe, thus other components such as sealing assembly are omitted.

An embodiment of the present disclosure provides a respiratory device.

The respiratory device includes the above check assembly and a respiratory isolation mask, and an end of the flow guide pipe is connected to an inhalation port of the respiratory isolation mask.

The check assembly is used in the respiratory device, may avoid the exhaled exhaust gas from being inhaled again, and also may avoid the outside air from being inhaled, so as to effectively reduce the influence that the exhaled exhaust gas or the outside air is mixed in the supply gas, improve the purity of the supply gas inhaled, and further effectively ensure the function and effect of the respiratory device. The check assembly may be set according to the shape and size of the respiratory isolation mask to facilitate installation and use of the check assembly.

### BRIEF DESCRIPTION OF DRAWINGS

In order to more clearly illustrate technical solutions of embodiments of the present disclosure, accompanying drawings which need to be used in the embodiments will be introduced briefly below, and it should be understood that the accompanying drawings below merely show some embodiments of the present disclosure, therefore, they should not be considered as limitation on the scope, and those ordinarily skilled in the art still could obtain other relevant accompanying drawings according to these accompanying drawings, without using any creative efforts.

FIG. 1 shows a schematic view of an internal structure of a check assembly provided in an embodiment of the present disclosure;

FIG. 2 shows a schematic view of an internal structure of a first embodiment of a fluid unidirectional flow structure, in a first state, provided in an embodiment of the present disclosure;

FIG. 3 shows a schematic view of the internal structure of the first embodiment of the fluid unidirectional flow structure, in a second state, provided in an embodiment of the present disclosure;

FIG. 4 shows a structural schematic view of the first embodiment of the fluid unidirectional flow structure, from another perspective, provided in an embodiment of the present disclosure;

FIG. 5 shows a schematic view of an internal structure of a second embodiment of the fluid unidirectional flow structure, in a first state, provided in an embodiment of the present disclosure;

FIG. 6 shows a schematic view of the internal structure of the second embodiment of the fluid unidirectional flow structure, in a second state, provided in an embodiment of the present disclosure;

FIG. 7 shows a schematic view of an internal structure of a third embodiment of the fluid unidirectional flow structure, in a first state, provided in an embodiment of the present disclosure;

FIG. 8 shows a schematic view of the internal structure of the third embodiment of the fluid unidirectional flow structure, in a second state, provided in an embodiment of the present disclosure;

FIG. 9 shows a schematic view of the internal structure of the check assembly, in a first state, provided in an embodiment of the present disclosure;

FIG. 10 shows a schematic view of the internal structure of the check assembly, in a second state, provided in an embodiment of the present disclosure.

FIG. 11 shows a structural schematic view of a respiratory isolation mask, in a first state, provided in an embodiment of the present disclosure.

FIG. 12 shows a structural schematic view of the respiratory isolation mask, in a second state, provided in an embodiment of the present disclosure.

Reference signs: 10—check assembly; 11—flow guide pipe; 12—first chamber; 13—second chamber; 14—installation hole; 100—fluid unidirectional flow structure; 101—gap; 102—cavity; 110—first flow-checking body; 111—first connection portion; 112—first flow portion; 113—first through-hole; 120—second flow-checking body; 121—second connection portion; 122—second flow portion; 123—second through-hole; 130—adjusting member; 131—toggle block; 20—check assembly; 1000—respiratory isolation mask.

### DETAILED DESCRIPTION OF EMBODIMENTS

In order to make objectives, technical solutions, and advantages of the embodiments of the present disclosure clearer, the technical solutions in the embodiments of the present disclosure will be described clearly and completely below in conjunction with accompanying drawings in the embodiments of the present disclosure, and apparently, the embodiments described are some but not all embodiments of the present disclosure. Generally, components in the embodiments of the present disclosure, as described and shown in the accompanying drawings herein, may be arranged and designed in various different configurations.

Therefore, the detailed description below of the embodiments of the present disclosure provided in the accompanying drawings is not intended to limit the scope of the present disclosure claimed, but merely illustrates chosen embodiments of the present disclosure. All other embodiments obtained by those ordinarily skilled in the art based on the embodiments of the present disclosure without any creative efforts shall fall within the scope of protection of the present disclosure.

It should be noted that similar reference signs and letters represent similar items in the following accompanying drawings, therefore, once a certain item is defined in one accompanying drawing, it is not needed to be further defined or explained in subsequent accompanying drawings.

In the description of the embodiments of the present disclosure, it should be understood that orientation or positional relationships indicated by terms such as “center”, “upper”, “lower”, “left”, “right”, “vertical”, “horizontal”, “inner”, and “outer” are based on orientation or positional relationships as shown in the accompanying drawings, or orientation or positional relationships of a product of the present disclosure when being conventionally placed in use, merely for facilitating describing the present disclosure and simplifying the description, rather than indicating or suggesting that related devices or elements have to be in the specific orientation or configured and operated in a specific orientation, therefore, they should not be construed as limitation to the present disclosure.

Besides, terms such as “first” and “second” are merely used for distinctive description, but should not be construed as indicating or implying importance in the relativity.

In the description of the embodiments of the present disclosure, it further needs to be indicated that unless otherwise specified and defined explicitly, terms “provide”, “mount”, and “connect” should be construed in a broad sense. For example, it may be a fixed connection, and also may be a detachable connection, or an integrated connection; it may be a direct connection, and also may be an indirect connection through an intermediary, or inner communication between two elements. For those ordinarily skilled in the art, specific meanings of the above-mentioned terms in the present disclosure could be understood according to specific circumstances.

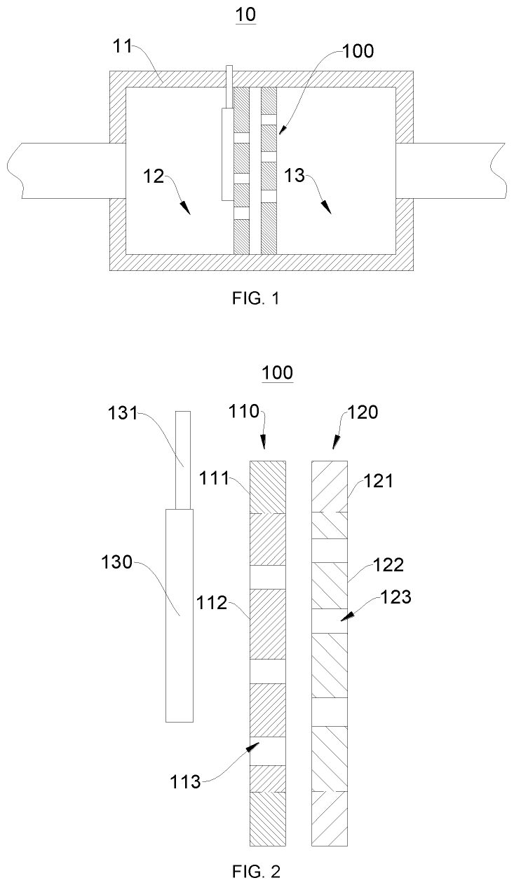

FIG. 1 shows a schematic view of an internal structure of a check assembly 10 provided in an embodiment of the present disclosure, referring to FIG. 1. An embodiment of the present disclosure provides a check assembly 10, and the check assembly 10 mainly functions to allow a fluid or a part of a fluid to pass unidirectionally. In an embodiment of the present disclosure, the check assembly 10 is mainly used for a respiratory isolation mask. In the embodiment of the present disclosure, the respiratory isolation mask should be understood in a broad sense, including, but not limited to, a face mask, a mask, a respiratory mask, and the like. The fluid flowing through the check assembly 10 may be a gas, optionally a gas for being inhaled by a person or an animal or a gas exhaled thereby.

In an embodiment of the present disclosure, the check assembly 10 may be used for a respiratory device such as a gas mask, a respiratory isolation mask, an oxygen respiratory machine, and diving apparatus, and may also be used for manufacturing a check valve for industrial use to be installed in a fluid conveying pipeline, etc. Correspondingly, the fluid flowing through the check assembly 10 may be other gases (for example, oxygen gas, nitrogen gas or carbon dioxide), or may be a liquid, a gas-liquid mixture, a gas-solid mixture and so on. The present disclosure does not define the use of the check assembly 10 or the type of the fluid.

The check assembly 10 may include a flow guide pipe 11 and a fluid unidirectional flow structure 100; the fluid unidirectional flow structure 100 may be installed in the flow guide pipe 11, the fluid unidirectional flow structure 100 may include a first flow-checking body and a second flow-checking body that can be separated from and attached to each other, outer edges of the first flow-checking body and the second flow-checking body may be closely attached to an inner wall of the flow guide pipe 11, the first flow-checking body may be provided with first through-holes, and the second flow-checking body may be provided with second through-holes, and the first through-holes and the second through-holes may be staggered from each other. When the first flow-checking body and the second flow-checking body are attached to each other, the first through-holes may be blocked by an area of the second flow-checking body where no through-hole is provided, and the second through-holes may be blocked by an area of the first flow-checking body where no through-hole is provided, so that the entire fluid unidirectional flow structure 100 may separate the flow guide pipe 11 into a first chamber 12 and a second chamber 13 which are isolated from each other and impermeable to fluid; when the first flow-checking body and the second flow-checking body are separated from each other, a gap may be formed between the first flow-checking body and the second flow-checking body, the fluid in the first chamber 12 may enter the gap through the first through-holes in the first flow-checking body, and flow into the second chamber 13 through the second through-holes of the second flow-checking body, and alternatively, the fluid in the second chamber 13 may enter the gap through the second through-holes in the second flow-checking body, and flow into the first chamber 12 through the first through-hole in the first flow-checking body. Hence, the fluid unidirectional flow structure 100 can enable the first chamber 12 and the second chamber 13 to be in an isolated or communicated state by actions of attaching and separating the first flow-checking body and the second flow-checking body.

In other words, the fluid unidirectional flow structure 100 may be provided in the flow guide pipe 11, whether the first chamber 12 and the second chamber 13 are in communication or not depends upon actions of opening and closing the fluid unidirectional flow structure 100. The first chamber 12 and the second chamber 13 are in communication when the fluid unidirectional flow structure 100 is opened, that is, when the first flow-checking body and the second flow-checking body are separated; and the first chamber 12 and the second chamber 13 are isolated from each other when the fluid unidirectional flow structure 100 is closed, that is, when the first flow-checking body and the second flow-checking body are attached to each other.

In the present disclosure, the flow guide pipe 11 may be shaped as a circular pipe, a square pipe, a multi-prismatic pipe, or in any other shape having a cavity.

Several of multiple embodiments of the fluid unidirectional flow structure 100 provided in the present disclosure are described below.

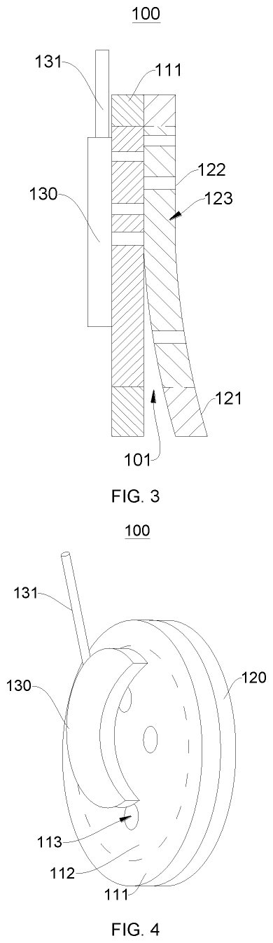

FIG. 2 shows a schematic view of an internal structure of a first embodiment of the fluid unidirectional flow structure 100, in a first state, provided in an embodiment of the present disclosure, and FIG. 3 shows a schematic view of the internal structure of the first embodiment of the fluid unidirectional flow structure 100, in a second state, provided in an embodiment of the present disclosure. Reference is made to FIG. 2 and FIG. 3.

The fluid unidirectional flow structure 100 may include a first flow-checking body 110 and a second flow-checking body 120.

The first flow-checking body 110 may include a first connection portion 111 and a first flow portion 112 hermetically connected to each other, and the first flow portion 112 may be provided with at least one first through-hole 113.

The second flow-checking body 120 may include a second connection portion 121 and a second flow portion 122 hermetically connected to each other, and the second flow portion 122 may be provided with at least one second through-hole 123.

In an embodiment of the present disclosure, the first through-hole 113 and the second through-hole 123 may be in a suitable shape and dimension.

Both the first through-hole 113 and the second through-hole 123 can allow a fluid to pass therethrough. The first connection portion 111 and the first flow portion 112 may be integrally arranged, or may be hermetically connected to each other by bonding or clamping etc. In an embodiment of the present disclosure, the first connection portion 111 may be provided at an outer edge of the first flow portion 112, the second connection portion 121 may be provided at an outer edge of the second flow portion 122, then the fluid cannot flow through the first connection portion 111 and the second connection portion 121. On the contrary, the first connection portion 111 may block the fluid on the outer edge of the first flow portion 112, and the second connection portion 121 may block the fluid on the outer edge of the second flow portion 122, in this way, under the effect of thrust of the fluid, the first flow-checking body 110 and the second flow-checking body 120 may be stressed more uniformly, preventing the first flow-checking body 110 and the second flow-checking body 120 from skewing or tipping along a flowing direction of the fluid. In addition, the outer edge of the first connection portion 111 may be closely attached to the inner wall of the flow guide pipe 11 without a gap, and the outer edge of the second connection portion 121 may be closely attached to the inner wall of the flow guide pipe 11 without a gap.

In an embodiment of the present disclosure, the first connection portion 111 may be provided at the outer edge of the first flow portion 112. It should be noted that, in an embodiment of the present disclosure, the first connection portion 111 may not be provided at the outer edge of the first flow portion 112, for example, the first flow portion 112 may be located at the outer edge of the first connection portion 111. Similarly, positional relationship between the second connection portion 121 and the second flow portion 122 may also be in other forms, which are not described herein again.

The first flow-checking body 110 and the second flow-checking body 120 may be independent from each other but used in cooperation with each other, and when the fluid flows in a different direction, that is, when the fluid changes from forward flowing to reverse flowing or from reverse flowing to forward flowing, at least a part of the first flow portion 112 and at least a part of the second flow portion 122 can move relative to each other. In other words, when the fluid flows in a different direction, the entire first flow portion 112 and the entire second flow portion 122 can be close to each other or away from each other, or only a part of the first flow portion 112 and a part of the second flow portion 122 are close to each other or away from each other.

Optionally, the first flow portion 112 may be fixedly connected in the flow guide pipe 11, the second flow portion 122 may be slidably connected in the flow guide pipe 11, and when the fluid flows forward, the fluid passes through the first through-hole 113 in the first flow portion 112 and pushes the second flow portion 122 in a forward direction, so that the first flow portion 112 and the second flow portion 122 may be partially or completely separated, thus, a gap 101 may be formed between the first flow portion 112 and the second flow portion 122, and the gap 101 may be in communication with both at least one first through-hole 113 and at least one second through-hole 123.

As shown in FIG. 2, when the fluid flows forward, the fluid enters the first through-holes 113, and as the first through-holes 113 and the second through-holes 123 are staggered with each other, the fluid entering the first through-holes 113 impacts an area of the second flow-checking body 120 which is corresponding to positions of the first through-holes 113 and where no through-hole is provided. Under the effect of thrust of the fluid, the second flow-checking body 120 is pushed away from the first flow-checking body 110 in a forward direction, thus the gap 101 is formed between the first flow portion 112 and the second flow portion 122. It should be noted that the gap 101 may extend over an entire contact surface area when the first flow portion 112 and the second flow portion 122 are attached to each other, or may merely extend over a part of the contact surface area when the first flow portion 112 and the second flow portion 122 are attached to each other. The gap 101 may be in communication with at least one first through-hole 113 and at least one second through-hole 123 to enable the fluid to flow through the first through-hole 113, the gap 101, and the second through-hole 123. In this case, the first chamber 12 and the second chamber 13 are in communication with each other.

Optionally, when the fluid flows reversely, the fluid pushes the second flow portion 122 in the reverse direction, so that the second flow portion 122 may be closely attached to the first flow portion 112, and all the first through-holes 113 may be blocked by the area of the second flow-checking body 120 where no through-hole is provided, and/or all the second through-holes 123 may be blocked by the area of the first flow-checking body 110 where no through-hole is provided.

As shown in FIG. 3, when the fluid flows reversely, the fluid may impact the area of the second flow portion 122 where no through-hole is provided, and may push the second flow portion 122 to get close to and finally be attached to the first flow portion 112 in the reverse direction, in this case, all the first through-holes 113 may be blocked by the area of the second flow-checking body 120 where no through-hole is provided, and/or all the second through-holes 123 may be blocked by the area of the first flow-checking body 110 where no through-hole is provided, thus, the fluid cannot flow from the second through-hole 123 to the first through-hole 113. In this case, the first chamber 12 and the second chamber 13 are isolated from each other.

Optionally, when the forward flowing of the fluid is switched to the reverse flowing of the fluid, at least a part of the first flow portion 112 and at least a part of the second flow portion 122 can move relative to each other, so that all the first through-holes 113 are blocked by the second flow-checking body 120; alternatively, all the second through-holes 123 are blocked by the first flow-checking body 110; alternatively, all the first through-holes 113 and all the second through-holes 123 are blocked, in this case, the first chamber 12 and the second chamber 13 are not in communication with each other.

In an embodiment of the present disclosure, the first through-hole 113 may be provided in the first flow-checking body 110, and the second through-hole 123 may be provided in the second flow-checking body 120, and unidirectional flowing may be realized through relative movement between at least a part of the first flow portion 112 and at least a part of the second flow portion 122.

The fluid unidirectional flow structure 100 may be designed in an arbitrary shape according to the material and shape of the flow guide pipe 11, so as to facilitate installation, and the material of the fluid unidirectional flow structure 100 may be flexible or rigid, and may or may not be elastic. The fluid unidirectional flow structure 100 may be installed on the flow guide pipe 11 in any shape. When the cross section of the flow guide pipe 11 is in an irregular shape, the fluid unidirectional flow structure 100 may also be installed, without increasing the installation difficulty and the volume of the flow guide pipe 11.

When the fluid unidirectional flow structure 100 is applied to a respiratory device, for example, when the flow guide pipe 11 provided with the fluid unidirectional flow structure 100 is installed in an inhalation tube or an respiratory tube, the fluid unidirectional flow structure 100 may be opened and closed under the effect of respiration of a user: when the user inhales, the fluid unidirectional flow structure 100 is closed, i.e. when the first flow-checking body and the second flow-checking body are attached to each other, outside unpurified air may be prevented from being inhaled by the user; when the user exhales, the fluid unidirectional flow structure 100 is opened, i.e. when the first flow-checking body and the second flow-checking body are separated, it may be convenient to discharge exhaled exhaust gas and prevent the user from re-inhaling the exhaled exhaust gas, thereby effectively reducing mixed influence of exhaled exhaust gas or outside air on the supply gas, improving the purity of the supply gas inhaled, and further more effectively ensuring the function and effect of the respiratory device.

Respiratory tubes have different shapes and different dimensions, but the fluid unidirectional flow structure 100 may be applicable to all cases. In addition, the fluid unidirectional flow structure 100 may also be installed on a respiratory isolation mask body, wherein the respiratory isolation mask body has a shape adapted to a face shape of a person or an animal, and the first flow-checking body 110 and the second flow-checking body 120 may be set to adapted shapes, without affecting the installation and use of the fluid unidirectional flow structure 100.

In the first embodiment, referring to FIG. 3, when the fluid flows reversely, two opposite surfaces of the first flow portion 112 and the second flow portion 122 are partially attached to each other, and there may still be a gap between remaining portions of the two surfaces, but the gap is in communication with none of the first through-holes 113 and none of the second through-holes 123.

In other words, in this embodiment, the reverse flowing of the fluid drives the first flow portion 112 and the second flow portion 122 to move relative to each other, then two opposite surfaces of the first flow portion 112 and the second flow portion 122 may be partially attached to each other, that is, there is still a gap between the two surfaces, but this gap is in communication with none of the first through-holes 113 and none of the second through-holes 123. In this case, it may be realized that the first chamber 12 and the second chamber 13 are not in communication with each other. After the second flow-checking body 120 and the first flow-checking body 110 are attached to each other, the first through-holes 113 and the second through-holes 123 may be arranged in a manner of being “staggered” with each other, and further the fluid unidirectional flow structure 100 achieves the purpose of non-return.

Due to factors such as stress of the fluid and the shape of the first flow portion 112, the first flow-checking body 110 and the second flow-checking body 120 may be formed into an arbitrary shape in the relatively narrow and compact flow guide pipe 11, and when the fluid flows reversely, the two opposite surfaces of the first flow portion 112 and the second flow portion 122 may not be completely coincidentally attached to each other, but it is feasible as long as the first through-holes 113 and the second through-holes 123 are not in communication with each other.

The first flow-checking body 110 and the second flow-checking body 120 in this embodiment may be provided according to specific situations, and are not limited to the case where two opposite surfaces of the first flow-checking body 110 and the second flow-checking body 120 are both planes that may be attached to each other. The fluid unidirectional flow structure 100 may be adapted to a case where the shapes and dimensions of the first chamber 12 and the second chamber 13 are quite different.

To sum up, when the fluid flows in a different direction, at least a part of the first flow portion 112 and at least a part of the second flow portion 122 can move relatively.

In the present embodiment, when the fluid flows in a different direction, the first flow-checking body 110 and the second flow-checking body 120 can slide relative to each other, so that the first flow-checking body 110 and the second flow-checking body 120 can get close to each other or be away from each other, thus it is realized that at least a part of the first flow portion 112 and at least a part of the second flow portion 122 can move relatively.

Optionally, in the present embodiment, the first flow-checking body 110 and the second flow-checking body 120 can slide relative to each other at least in a following manner:

First, a slide rail or a slide groove may be provided on a side of the first flow-checking body 110 facing the second flow-checking body 120, and the second flow-checking body 120 may be slidably connected to the above slide rail or slide groove. In other words, in the first embodiment, the slide rail or the slide groove is provided at the side of the first flow-checking body 110.

Second, the first flow-checking body 110 may be fixedly connected to the flow guide pipe 11, and a slide rail or a slide groove may be provided in the flow guide pipe 11, and the second flow-checking body 120 may be slidably connected to the above slide rail or slide groove.

Third: the first flow-checking body 110 may be fixedly connected to the flow guide pipe 11, and a limiting block protruding along a radial direction may be provided inside the flow guide pipe 11, and the second flow-checking body 120 may be provided between the limiting block and the first flow-checking body 110, and the second flow-checking body 120 can slide between the limiting block and the first flow-checking body 110 under the effect of direction change of the fluid.

When the fluid flows in a different direction, the first flow-checking body 110 and the second flow-checking body 120 may be enabled to slide relative to each other due to the change of direction of a force applied on the them after the fluid is changed in direction, so as to achieve the purpose that at least a part of the first flow portion 112 and at least a part of the second flow portion 122 can move relative to each other.

FIG. 4 shows a structural schematic view of the first embodiment of the fluid unidirectional flow structure, from another perspective, provided in an embodiment of the present disclosure.

Referring to FIG. 1, FIG. 2, and FIG. 4, in the present disclosure, the fluid unidirectional flow structure 100 further may include an adjusting member 130; the adjusting member 130 may be movably connected to the first flow-checking body 110 such that a part of cross section of at least one first through-hole 113 can be covered by the adjusting element 130.

The adjusting member 130 can cover a part of cross section of at least one first through-hole 113, and correspondingly, the adjusting member 130 can cover a part of the first through-holes 113, and may also cover all the first through-holes 113. Thus, the adjusting member 130 may adjust magnitude of a flow rate passing through the first flow portion 112.

When the fluid unidirectional flow structure 100 is applied to a respiratory device, the adjusting member 130 may adjust a volume of the supply gas entering nose and mouth, for example, all the first through-holes 113 are opened during exercise, and an amount of gas is adjusted to a maximum, and the amount of gas may be reduced when sitting or lying down. It is convenient to make adjustment, and the comfort degree is increased.

Optionally, in the present embodiment, the adjusting member 130 is provided at least in a following manner:

The adjusting member 130 may be rotatably connected to a side of the first flow-checking body 110 away from the second flow-checking body 120 and be closely attached to a surface of the first flow-checking body 110, and the adjusting member 130 may be rotated about an axis passing through a centroid of a cross section of the first flow-checking body 110 transverse to the flowing direction of the fluid; for example, the adjusting member 130 is set as a profiled member (for example, in a crescent shape), and a cross section of the adjusting member 130 may be sized smaller than a cross section of the first flow-checking body 110. After the adjusting member 130 is rotated, the adjusting member 130 can block a part of at least one first through-hole 113. Alternatively, the adjusting member 130 may also be provided with a plurality of holes, and the plurality of holes can allow the fluid to flow therethrough. After the adjusting member 130 is rotated, the holes of the adjusting member 130 may be in communication with a part of at least one first through-hole 113, and the adjusting member 130 is allowed to block a part of at least one first through-hole 113. The cross section of the adjusting member 130 may be sized smaller than or equal to the cross section of the first flow-checking body 110.

It is also possible to provide an adjusting member slide rail on a wall of the flow guide pipe 11, the adjusting member 130 is slidably connected to the above adjusting member slide rail, and the adjusting member 130 may slide along the flowing direction of the fluid to be attached to the first flow-checking body 110, thereby achieving the purpose of at least blocking a part of one first through-hole 113; correspondingly, the cross section of the adjustment member 130 may be sized smaller than or equal to the dimension of the first flow-checking body 110.

In an embodiment of the present disclosure, the adjusting member 130 may be provided with a toggle block 131 protruding in a radial direction. The toggle block 131 may extend out of the flow guide pipe 11, and the above rotation or sliding of the adjusting member 130 relative to the first flow-checking body 110 may be actuated by toggling the toggle block 131. The toggle block 131 may be toggled manually or by a controller.

In an embodiment of the present disclosure, the adjusting member 130 may be provided on a side of the first flow-checking body 110 away from the second flow-checking body 120. It may be understood that, in an embodiment of the present disclosure, the adjusting member 130 may also be provided on a side of the first flow-checking body 110 facing the second flow-checking body 120.

In addition, in an embodiment of the present disclosure, the adjusting member 130 may also be provided to be movably connected to the second flow-checking body 120, and for the corresponding connection relationship, reference is made to the movable connection between the adjusting member 130 and the first flow-checking body 110.

In an embodiment of the present disclosure, the adjusting member 130 is not indispensable, that is, the adjusting member 130 may not be provided.

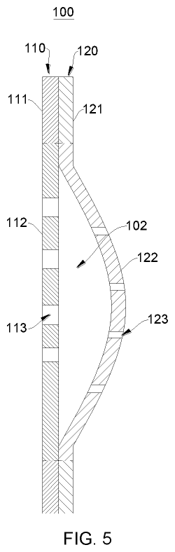

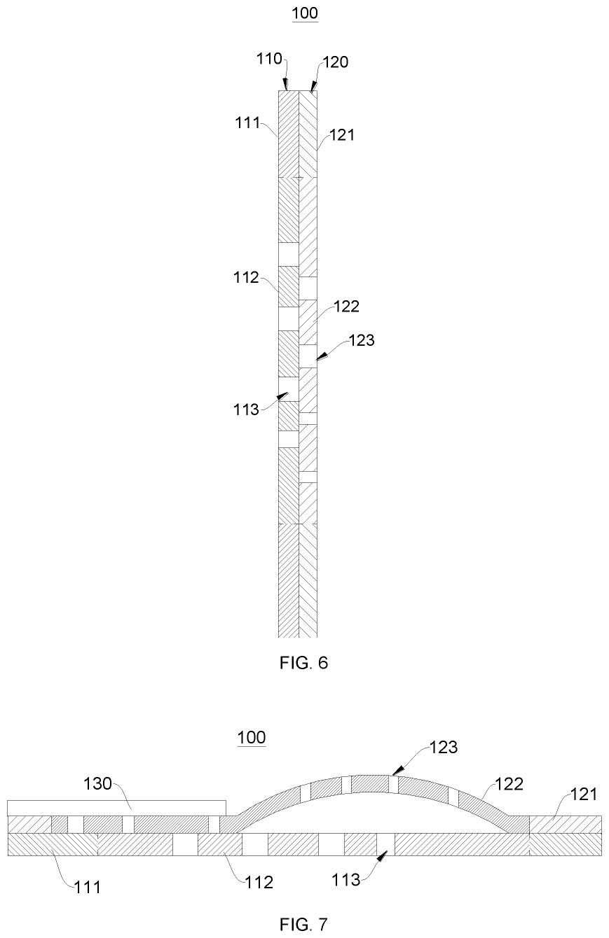

FIG. 5 shows a schematic view of an internal structure of a second embodiment of the fluid unidirectional flow structure 100, in a first state, provided in an embodiment of the present disclosure, and FIG. 6 shows a schematic view of the internal structure of the second embodiment of the fluid unidirectional flow structure 100, in a second state, provided in an embodiment of the present disclosure.

Referring to FIG. 2 to FIG. 6, a first difference between the fluid unidirectional flow structure 100 provided in the present embodiment and the fluid unidirectional flow structure 100 provided in the first embodiment lies in connection relationship between the first flow-checking body 110 and the second flow-checking body 120.

In the present embodiment, the first flow-checking body 110 may be connected to the second flow-checking body 120, and optionally, in the present embodiment, the first connection portion 111 may be connected to the second connection portion 121, for example, by bonding or grasping, and the first connection portion 111 may not be in communication with the second connection portion 121 at a junction.

When the fluid flows in a different direction, at least a part of the first flow portion 112 and at least a part of the second flow portion 122 can move relative to each other.

The first flow portion 112 and the second flow portion 122 may have at least the following embodiments.

First, the first flow portion 112 and the second flow portion 122 may be both of a flexible material, such as polyethylene, and the second flow portion 122 may have a larger surface area than the first flow portion 112. When the fluid flows forward, under the effect of the fluid, the first flow portion 112 and the second flow portion 122 may have a cavity 102 therebetween, further enabling the first chamber 12 and the second chamber 13 to be in communication. When the fluid flows reversely, under the effect of the fluid, the second flow portion 122 may be attached to the first flow portion 112, and the second flow portion 122 blocks all the first through-holes 113 of the first flow portion 112, and the first chamber 12 and the second chamber 13 are not in communication with each other.

Second: both the first flow portion 112 and the second flow portion 122 may be both made of an elastic material, and an elastic modulus of the first flow portion 112 may be greater than that of the second flow portion 122; when the fluid flows forward, the first flow portion 112 and the second flow portion 122 may be subjected to the same force, but deformed to different degrees, wherein deformation of the second flow portion 122 is greater than that of the first flow portion 112, and the cavity 102 will be formed between the first flow portion 112 and the second flow portion 122, further the first chamber 12 and the second chamber 13 are in communication. When the fluid flows reversely, under the effect of the fluid, the first flow portion 112 and the second flow portion 122 are attached to each other. The first chamber 12 and the second chamber 13 are not in communication with each other.

The first flow portion 112 and the second flow portion 122 may have elasticity, which may make the two more flexible, and when used in a respiratory device, may increase the comfort of the respiratory device.

In addition, the first chamber 12 and the second chamber 13 may be in any shape, and under the effect of the fluid, both the first flow portion 112 and the second flow portion 122 can be deformed to achieve the function of opening and closing, and the first flow portion 112 and the second flow portion 122 of the elastic material are easily provided, and occupy a small space.

Third: the first flow portion 112 may be made of an inelastic material, and the second flow portion 122 may be made of an elastic material. When the fluid flows forward, the second flow portion 122 may be deformed, and the cavity 102 will be formed between the first flow portion 112 and the second flow portion 122, thereby enabling communication between the first chamber 12 and the second chamber 13. When the fluid flows reversely, the second flow portion 122 is restored, and the first flow portion 112 and the second flow portion 122 are attached to each other. The first chamber 12 and the second chamber 13 are not in communication with each other.

Fourth: the first flow portion 112 may be made of a rigid material, such as stainless steel, aluminum, copper, nickel, plastic, ABS, alloy, medical plastic, carbon fiber, organic glass, glass, ceramic, or polyurethane flexible material; the second flow portion 122 may be made of a flexible material, such as resin film, rubber, fabric coated with an impermeable coating, silicone, latex, PVC, thermoplastic rubber, mixed rubber, or TPE material; when the fluid flows forward, the second flow portion 122 may be away from the first flow portion 112 under the effect of the fluid, and the cavity 102 will be formed between the first flow portion 112 and the second flow portion 122, as shown in FIG. 5, the first chamber 12 and the second chamber 13 are in communication. When the fluid flows reversely, the second flow portion 122 may be attached to the first flow portion 112 under the effect of the fluid, as shown in FIG. 6, the first chamber 12 and the second chamber 13 are not in communication with each other.

The first flow portion 112 and the second flow portion 122 shown in FIG. 5 and FIG. 6 may be circular and have a relatively regular surface. In an embodiment of the present disclosure, the surfaces of the first flow portion 112 and the second flow portion 122 may be planes or may be curved surfaces.

A second difference between the fluid unidirectional flow structure 100 provided in the present embodiment and the fluid unidirectional flow structure 100 provided in the first embodiment lies that the fluid unidirectional flow structure 100 in the present embodiment is not provided with the adjusting member 130.

It should be noted that, in other embodiments of the present disclosure, the fluid unidirectional flow structure 100 may have the structures of both the first embodiment and the second embodiment without mutual conflict, and the two are not merely alternative, for example, the slidable connection in the first embodiment and the first flow portion 112 and the second flow portion 122 being elastic in the second embodiment may be simultaneously provided in a fluid unidirectional flow structure 100.

The above are several of multiple embodiments of the fluid unidirectional flow structure 100 provided in the embodiments of the present disclosure.

To sum up, the fluid unidirectional flow structure 100 may be provided in the flow guide pipe 11, and in an embodiment of the present disclosure, the first connection portion 111 and the inner wall of the flow guide pipe 11 may be hermetically connected (for example, bonding, welding or integrally provided), in other words, the edge of the first flow-checking body 110 may not have a gap with the flow guide pipe 11. Installation is facilitated. When the fluid unidirectional flow structure 100 is closed, the fluid can be avoided from flowing between the edge of the first flow-checking body 110 and the flow guide pipe 11, and other components such as the sealing assembly are omitted.

In an embodiment of the present disclosure, a sealing ring may be provided between the first connection portion 111 and the flow guide pipe 11 for hermetic connection.

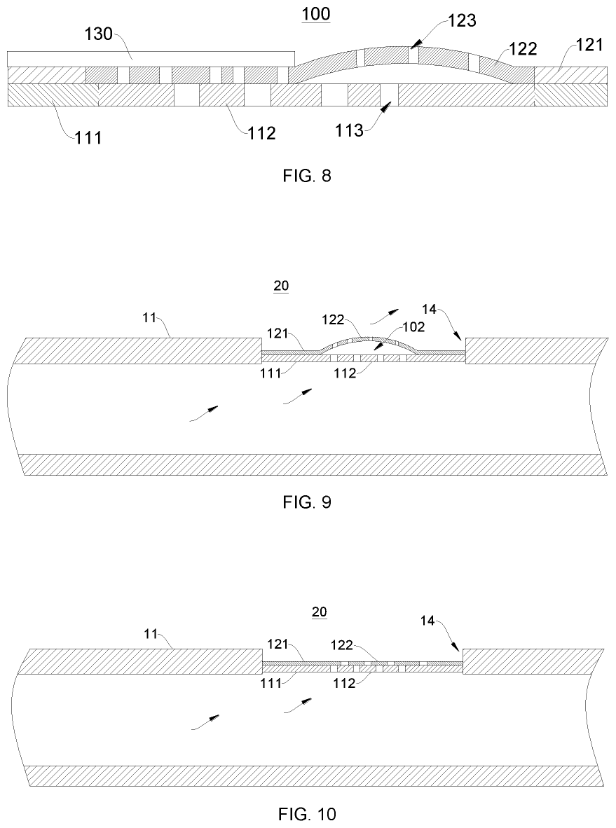

FIG. 7 shows a schematic view of an internal structure of a third embodiment of the fluid unidirectional flow structure 100, in a first state, provided in an embodiment of the present disclosure, and FIG. 8 shows a schematic view of the internal structure of the third embodiment of the fluid unidirectional flow structure 100, in a second state, provided in an embodiment of the present disclosure.

Referring to FIG. 5 to FIG. 8, a first difference between the fluid unidirectional flow structure 100 provided in the present embodiment and the fluid unidirectional flow structure 100 provided in the second embodiment lies in that in the present embodiment, the fluid unidirectional flow structure 100 further includes the adjusting member 130.

In the present embodiment, the adjusting member 130 may be movably connected to the second flow-checking body 120, for example, slidably connected by a slide groove, a slide block, a slide closure or the like.

The adjustment member 130 may be movably coupled to the second flow-checking body 120 to enable the adjustment member 130 to be capable of blocking a part of the cross section of at least one second through-hole 123.

For an embodiment in which the adjusting member 130 is movably connected to the second flow-checking body 120:

the adjusting member 130 may adjust the size of an area of acting force of the second flow portion 122 by adjusting the number of opened second through-holes 123 of the second flow portion 122, so that on the one hand, the difficulty degree of flowing can be adjusted so as to adjust the flow rate, and on the other hand, magnitude of stress of the second flow portion 122 can be adjusted so as to adjust the non-returning effect.

When a larger number of second through-holes 123 are blocked by the adjusting member 130, less gas can enable at least a part of the first flow portion 112 to move relative to at least a part of the second flow portion 122, so that the fluid unidirectional flow structure 100 is closed, and during the process of closing the fluid unidirectional flow structure 100, the amount of gas returned from the second through-holes 123 is small (for example, outside unpurified air), thereby increasing the non-returning effect, and decreasing the ventilation effect. When a smaller number of second through-holes 123 are blocked by the adjusting member 130, more gas is needed to enable at least a part of the first flow portion 112 to move relative to at least a part of the second flow portion 122, so that the fluid unidirectional flow structure 100 is closed, and during the process of closing the fluid unidirectional flow structure 100, the amount of gas returned from the second through-holes 123 is large, thereby decreasing the non-returning effect, and increasing the ventilation effect.

The check assembly 10 provided in the embodiments of the present disclosure at least has the following advantages.

The fluid unidirectional flow structure 100 may control communication and isolation between the first chamber 12 and the second chamber 13. The check assembly 10 has all the advantages of the fluid unidirectional flow structure 100. In the check assembly 10 provided in the embodiment of the present disclosure, the fluid unidirectional flow structure 100 is provided inside the flow guide pipe 11, so as to facilitate the installation of the fluid unidirectional flow structure 100.

With regard to the flow guide pipe 11 with an irregular cross section, in the prior art, the irregular cross section needs to be divided into a plurality of regular cross sections, and valve bodies are installed at the plurality of regular cross sections, thus inevitably increasing the cost and weight, and making the installation process troublesome. In an embodiment of the present disclosure, the fluid unidirectional flow structure 100 may be directly installed to the flow guide pipe 11 with an irregular cross section, without installing after procedures such as cutting the flow guide pipe in such a way that the irregular cross section is divided into regular cross sections.

FIG. 9 shows a schematic view of the internal structure of the check assembly 20, in a first state, provided in an embodiment of the present disclosure, and FIG. 10 shows a schematic view of the internal structure of the check assembly 20, in a second state, provided in an embodiment of the present disclosure.

An outer wall of the flow guide pipe 11 may be provided with an installation hole 14 running through the flow guide pipe 11, and the first flow-checking body 110 may be installed in the installation hole 14.

The installation hole 14 may run through the outer wall of the flow guide pipe 11, and the fluid unidirectional flow structure 100 may control whether the inside and outside of the flow guide pipe 11 are in communication, and further control whether to discharge the fluid inside the flow guide pipe 11, or prevent the fluid outside the flow guide pipe 11 from flowing into the flow guide pipe 11.

When the check assembly 20 is used in a respiratory device, for example, the check assembly 20 is installed in an inhalation tube, the flow guide pipe 11 may act as the inhalation tube, and the fluid unidirectional flow structure 100 is opened during exhalation, the gas exhaled from the inside of the flow guide pipe 11 is discharged, and the fluid unidirectional flow structure 100 is closed during inhalation, and the outside gas will not flow into the nose and mouth through the flow guide pipe 11.

In an embodiment of the present disclosure, the fluid unidirectional flow structure 100 may be provided inside the flow guide pipe 11, and in an embodiment of the present disclosure, the first connection portion 111 and the inner wall of the flow guide pipe 11 may be hermetically connected (for example, bonding, welding or integrally provided), in other words, the edge of the first flow-checking body 110 may not have a gap with the flow guide pipe 11. Installation is facilitated. When the fluid unidirectional flow structure 100 is closed, the fluid may be prevented from flowing between the edge of the first flow-checking body 110 and the flow guide pipe 11, so as to prevent fluid leakage, and other components such as the sealing assembly are omitted. In an embodiment of the present disclosure, a sealing ring may be arranged between the first connection portion 111 and the flow guide pipe 11 for hermetic connection.

The relative sliding between the first flow-checking body 110 and the second flow-checking body 120 requires providing a slide rail, a slide groove or a limiting block, and the slide rail, the slide groove or the limiting block demands a certain space. The first flow-checking body 110 and the second flow-checking body 120 may be connected to each other, and both the first flow portion 112 and the second flow portion 122 may be made of an elastic material, or the first flow portion 112 may be made of a rigid material, and the second flow portion 122 may be made of a flexible material. Space may be better saved.

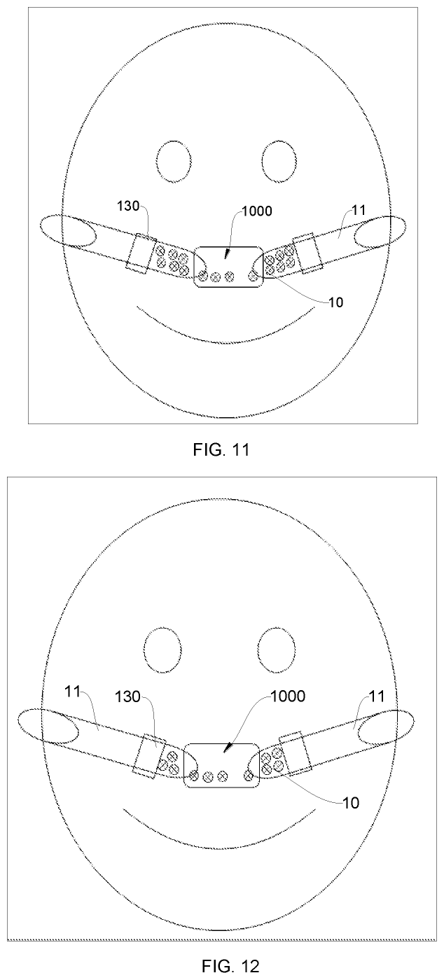

FIG. 11 shows a structural schematic view of a respiratory isolation mask 1000, in a first state, provided in an embodiment of the present disclosure; and FIG. 12 shows a structural schematic view of the respiratory isolation mask 1000, in a second state, provided in an embodiment of the present disclosure. Reference is made to FIG. 11 and FIG. 12. An embodiment of the present disclosure provides a respiratory isolation mask 1000. The respiratory isolation mask 1000 may include a mask body, inhalation pipe(s), and the check assemblies 20 provided in an embodiment of the present disclosure. The respiratory isolation mask 1000 may be connected to the inhalation pipes, and the check assemblies 20 may be installed on the inhalation pipe(s) (i.e., the flow guide pipe(s) 11 for conveying the supply gas to the respiratory isolation mask 1000).

Optionally, the check assembly 20 may be installed on an end of the inhalation pipe close to the body of the respiratory isolation mask 1000, which may reduce the volume of exhaled exhaust gas entering the inhalation pipe, avoid mixing of the exhaust gas and the supply gas as much as possible, and may prevent the exhaust gas from being inhaled again during re-inhalation.

In an embodiment of the present disclosure, the respiratory isolation mask 1000 may include two flow guide pipes 11 and a plurality of check assemblies 20; each of the flow guide pipes 11 may be provided with at least one check assembly 20, and correspondingly, the mask body may also be provided with one check assembly 20.

In an embodiment of the present disclosure, the respiratory isolation mask 1000 may be provided with only one check assembly 20, for example, provided on the body of the respiratory isolation mask 1000, and the respiratory isolation mask 1000 may also be provided with only one flow guide pipe 11.

Referring to FIG. 11 and FIG. 12, the adjusting member 130 in FIG. 12 may move (e.g., slide or rotate) relative to the flow guide pipe 11 to enable the adjusting member 130 to block at least a part of at least one first through-hole 113 or at least a part of at least one second through-hole 123 in the check assembly 20, so as to adjust the flow rate of the gas when the check assembly 20 is opened.

The check assembly 20, when used in the respiratory isolation mask 1000, may avoid the inhalation of outside air, so as to effectively reduce the mixed influence of the outside air on the supply gas, improve the purity of the supply gas inhaled, and further effectively ensure the function and effect of the respiratory device. The check assembly 20 may be set according to the shape and size of the respiratory isolation mask 1000 to facilitate installation and use of the check assembly 20.

An embodiment of the present disclosure provides a respiratory device, wherein the respiratory device includes the respiratory isolation mask 1000 and the check assembly 10 provided in the embodiments of the present disclosure.

In an embodiment of the present disclosure, the respiratory isolation mask 1000 may be connected to an inhalation pipe, and the check assembly 10 may be installed on the inhalation pipe (namely, a tube for the respiratory isolation mask 1000 to suck purified air) of the respiratory isolation mask 1000.

In an embodiment of the present disclosure, the respiratory isolation mask 1000 may include two flow guide pipes 11; and each of the flow guide pipes 11 may be provided with at least one check assembly 10.

In an embodiment of the present disclosure, the two flow guide pipes 11 may share one check assembly 10, for example, the two flow guide pipes 11 may be connected and merged into one manifold at one end away from the respiratory isolation mask 1000, and the check assembly 10 may be provided on the manifold.

The check assembly 10 is configured for the respiratory isolation mask 1000, and may avoid the exhaled exhaust gas from flowing back into the inhalation pipe to be inhaled again. The check assembly 10 may be set according to the shape and size of the respiratory isolation mask 1000 to facilitate installation and use of the check assembly 10.

The above-mentioned are merely for preferred examples of the present disclosure and not used to limit the present disclosure. For one skilled in the art, various modifications and changes may be made to the present disclosure. Any modifications, equivalent substitutions, improvements and so on, within the spirit and principle of the present disclosure, should be covered within the scope of protection of the present disclosure.

### INDUSTRIAL APPLICABILITY

To sum up, the present disclosure provides a fluid unidirectional flow structure, a check assembly, and a respiratory device, which may avoid the exhaled exhaust gas from being inhaled again, and also may avoid the outside air from being inhaled, so as to effectively reduce the mixed influence of the exhaled exhaust gas or the outside air on the supply gas, improve the purity of the supply gas inhaled, and further effectively ensure the function and effect of the respiratory device. The check assembly may be set according to the shape and size of the respiratory isolation mask to facilitate installation and use of the check assembly.

1. A fluid unidirectional flow structure, wherein the fluid unidirectional flow structure comprises:
a first flow-checking body and a second flow-checking body;
the first flow-checking body comprises a first connection portion and a first flow portion connected to each other, and the first flow portion is provided with at least one first through-hole;
the second flow-checking body comprises a second connection portion and a second flow portion connected to each other, and the second flow portion is provided with at least one second through-hole; and
when a fluid flows in a different direction, at least a part of the first flow portion and at least a part of the second flow portion are capable of moving relative to each other,
wherein when the fluid flows forward, a gap is formed between the first flow portion and the second flow portion, and the gap is in communication with the at least one first through-hole and the at least one second through-hole, and
when the fluid flows reversely, all of the at least one first through-hole is blocked by the second flow-checking body, and/or all of the at least one second through-hole is blocked by the first flow-checking body.

2. The fluid unidirectional flow structure according to claim 1, wherein the at least one first through-hole and the at least one second through-hole are arranged in a manner of being staggered with each other.

3. The fluid unidirectional flow structure according to claim 2, wherein when the fluid flows reversely, two opposite surfaces of the first flow portion and the second flow portion are partially attached to each other, a gap is formed between remaining portions of the two surfaces, and the gap is in communication with none of the at least one first through-hole and none of the at least one second through-hole.

4. The fluid unidirectional flow structure according to claim 3, wherein the first connection portion is provided at an outer edge of the first flow portion, and the second connection portion is provided at an outer edge of the second flow portion.

5. The fluid unidirectional flow structure according to claim 4, wherein the first flow-checking body and the second flow-checking body are slidable relative to each other, wherein when the fluid flows in a different direction, the first flow portion and the second flow portion are capable of being attached to or separated from each other.

6. The fluid unidirectional flow structure according to claim 5, wherein when the fluid flows reversely, two opposite surfaces of the first flow portion and the second flow portion are at least partially attached to each other; and the two surfaces are both curved surfaces or planes.

7. The fluid unidirectional flow structure according to claim 6, wherein an elastic modulus of the first flow portion is greater than an elastic modulus of the second flow portion.

8. The fluid unidirectional flow structure according to claim 7, wherein the flow unidirectional flow structure further comprises an adjusting member,
the adjusting member is movably connected to the first flow-checking body, and
a part of cross section of the at least one first through-hole is covered by the adjusting member; alternatively
the adjusting member is movably connected to the second flow-checking body,
and a part of cross section of the at least one second through-hole is covered by the adjusting member.

9. The fluid unidirectional flow structure according to claim 8, wherein the adjusting member is capable of sliding towards the first flow-checking body to be attached to the first flow-checking body, and the adjusting member is capable of sliding away from the first flow-checking body to be separated from the first flow-checking body; alternatively
the adjusting member is capable of being rotated about an axis passing through a centroid of a cross section of the first flow-checking body transverse to a flowing direction of the fluid, so as to cover a part of the cross section of the at least one first through-hole.

10. The fluid unidirectional flow structure according to claim 9, wherein the adjusting member is rotatably connected to a side of the first flow-checking body away from the second flow-checking body and closely attached to a surface of the first flow-checking body.

11. A check assembly, wherein the check assembly comprises a flow guide pipe and the fluid unidirectional flow structure according to claim 1; the fluid unidirectional flow structure is installed inside the flow guide pipe, the fluid unidirectional flow structure divides the flow guide pipe into a first chamber and a second chamber; and the fluid unidirectional flow structure enables the first chamber to be isolated from or in communication with the second chamber.

12. The check assembly according to claim 11, wherein the first flow-checking body is fixedly connected in the flow guide pipe, and the second flow-checking body is slidably connected in the flow guide pipe; alternatively,
the first flow-checking body and the second flow-checking body are both fixedly connected in the flow guide pipe, and the first flow-checking body and the second flow-checking body have different elastic moduli.

13. The check assembly according to claim 12, wherein an outer edge of the first flow-checking body is closely attached to an inner wall of the flow guide pipe.

14. The check assembly according to claim 13, wherein the flow guide pipe is provided therein with a slide rail or a slide groove having a preset length; and the second flow-checking body is slidably connected to the slide rail or the slide groove.

15. The check assembly according to claim 14, wherein the flow guide pipe is provided therein with a limiting block protruding in a radial direction, the first flow-checking body is fixedly connected to the flow guide pipe, the second flow-checking body is provided between the limiting block and the first flow-checking body, and the second flow-checking body is slidable between the limiting block and the first flow-checking body.

16. (canceled)

17. (canceled)

18. A respiratory device, wherein the respiratory device comprises the check assembly according to claim 11 and a respiratory isolation mask, and an end of the flow guide pipe is connected to an inhalation port of the respiratory isolation mask.

19. The fluid unidirectional flow structure according to claim 7, wherein the first flow portion is made of a rigid material, and the second flow portion is made of a flexible material.

20. The fluid unidirectional flow structure according to claim 1, wherein when the fluid flows reversely, two opposite surfaces of the first flow portion and the second flow portion are partially attached to each other, a gap is formed between remaining portions of the two surfaces, and the gap is in communication with none of the at least one first through-hole and none of the at least one second through-hole.

21. The fluid unidirectional flow structure according to claim 1, wherein the first connection portion is provided at an outer edge of the first flow portion, and the second connection portion is provided at an outer edge of the second flow portion.

22. The fluid unidirectional flow structure according to claim 2, wherein the first connection portion is provided at an outer edge of the first flow portion, and the second connection portion is provided at an outer edge of the second flow portion.
